(19) United States
(12) Patent Application Publication
(10) Pub. No.: US 2013/0296608 A1
(43) Pub. Date: Nov. 7, 2013

(54) NOVEL STEREOSPECIFIC SYNTHESIS OF (-) (2S, 3S)-1-DIMETHYLAMINO-3-(3-METHOXYPHENYL)-2-METHYL PENTAN-3-OL

(71) Applicants: Dodda Mohan Rao, Hyderabad (IN); Pingili Krishnareddy, Hyderabad (IN); Pingili Ramachandrareddy, Hyderabad (IN); Kirla Haritha, Pamarru (IN); Kolluru Srinivas, Gatkesar (IN)

(72) Inventors: Dodda Mohan Rao, Hyderabad (IN); Pingili Krishnareddy, Hyderabad (IN); Pingili Ramachandrareddy, Hyderabad (IN); Kirla Haritha, Pamarru (IN); Kolluru Srinivas, Gatkesar (IN)

(73) Assignee: SYMED LABS LIMITED, Hyderabad, Andhra Pradesh (IN)

(21) Appl. No.: 13/581,655

(22) PCT Filed: Jan. 27, 2011
(86) PCT No.: PCT/IN11/00054

§ 371 Date: Aug. 29, 2012

(51) Int. Cl. C07C 213/00; C07C 221/00

(52) U.S. Cl. CPC C07C 213/00; C07C 221/00
USPC 564/302; 564/355; 564/502

(57) ABSTRACT

The present invention relates to a novel stereospecific synthesis of (−)(2S,3S)-1-dimethylamino-3-(3-methoxyphenyl)-2-methyl pentan-3-ol an intermediate in the synthesis of 3-[(1R,2R)-3-(dimethylamino)-1-ethyl-2-methylpropyl]phenol.

### BACKGROUND OF THE INVENTION

1. Technical Field

The present invention relates to a novel stereospecific synthesis of (−)(2S,3S)-1-dimethylamino-3-(3-methoxyphenyl)-2-methyl pentan-3-ol an intermediate in the synthesis of tapentadol.

2. Description of the Related Art

Tapentadol is an analgesic which has been approved as tapentadol hydrochloride in US and EU for the treatment of acute pain and available in the market under the brand name Nucynta in the form of tablets in dosage strengths 50, 75 and 100 mg as equivalent to base. Tapentadol hydrochloride is chemically described as 3-[(1R,2R)-3-(dimethylamino)-1-ethyl-2-methylpropyl]phenol hydrochloride (herein after referred by its generic name tapentadol) and has the following structure (I):

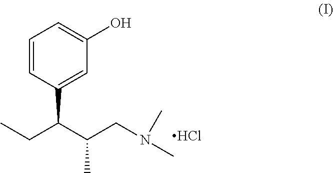 [SMILES: CC[C@@H](c1cccc(O)c1)[C@@H](C)CN(C)C ; Cl]

U.S. Pat. No. USRE39593E (U.S. Pat. No. 6,248,737B1) describes tapentadol and its related compound along with their pharmaceutically acceptable salts, a pharmaceutical composition and method of treatment. The US '593 patent discloses a process for the preparation of tapentadol hydrochloride, which is illustrated by the scheme below:

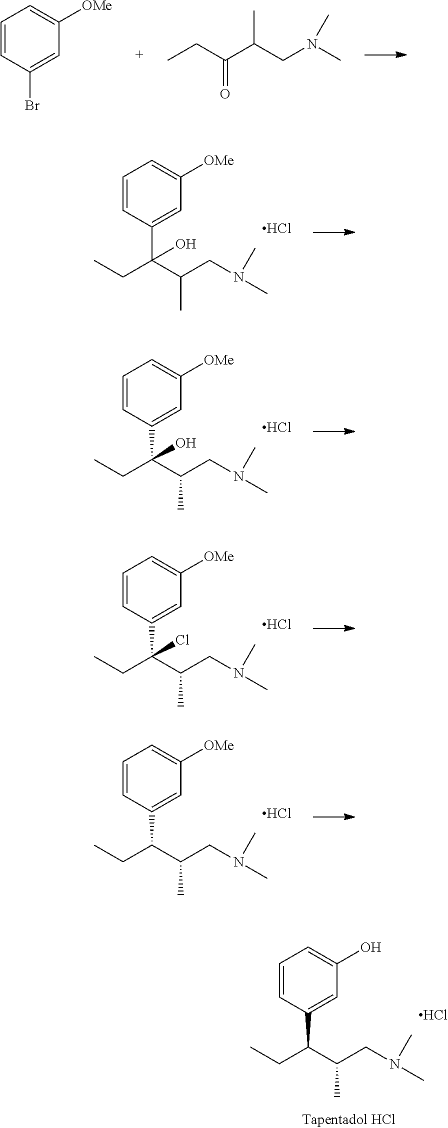

US application publication US20090326271A1 describes a process for the preparation of tapentadol hydrochloride which is illustrated by the below scheme

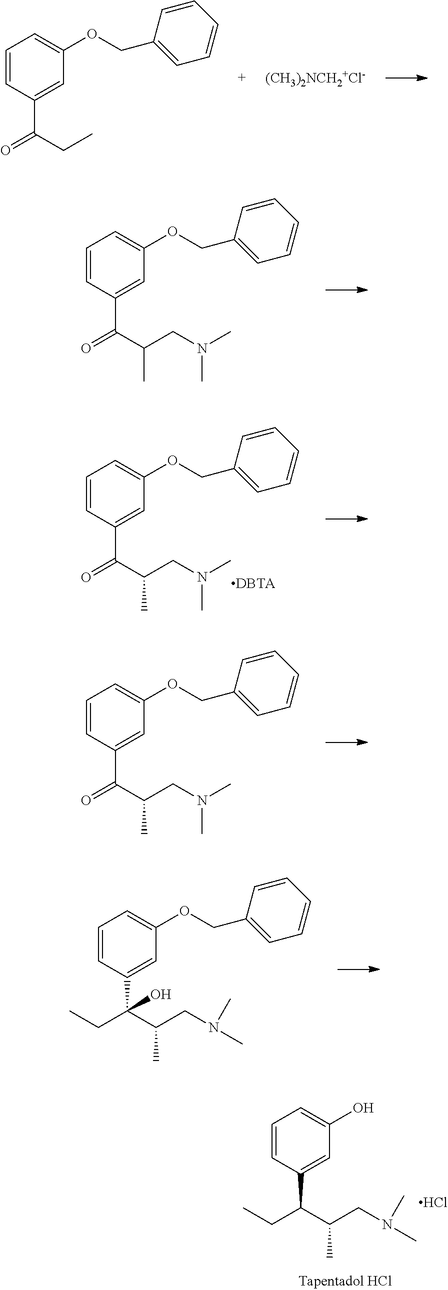

US application publication US2010099916A1 describes a process for the preparation of tapentadol hydrochloride which is illustrated by the below scheme

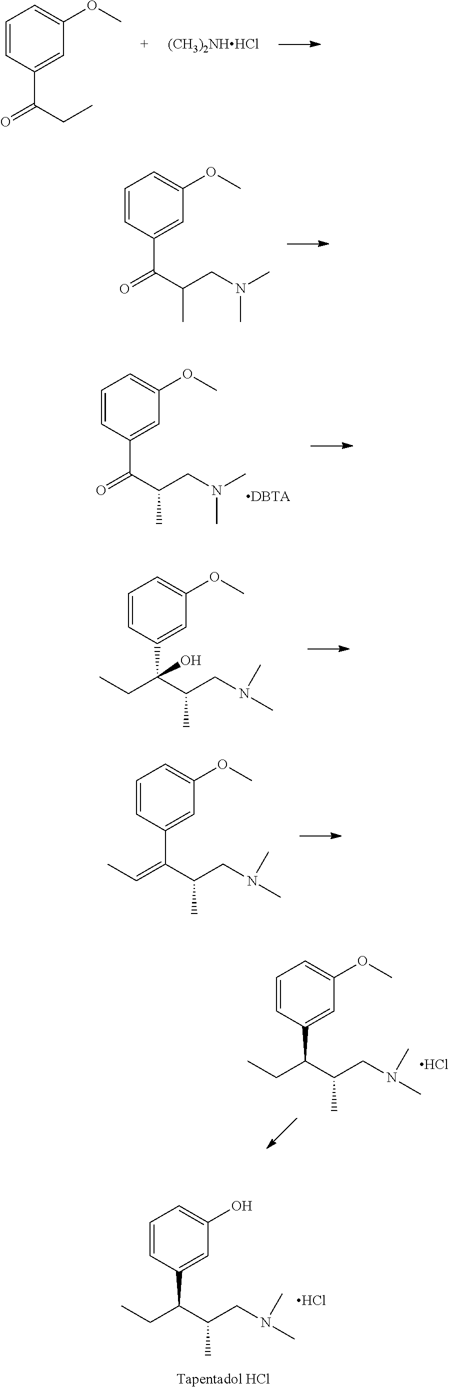

Aforementioned process for the preparation of intermediate compound of formula (III) involves separation of optical isomers by chiral HPLC, which is tedious, involves the use of larger volumes of organic solvents and results in very low yields thus making the process not ecofriendly and not suitable on commercial scale. Moreover the unwanted isomers cannot be racemised and recycled.

Hence, there is a need to provide a process that would avoid the aforementioned steps like chiral HPLC and that could be well applicable on commercial scale and also the unwanted isomers can be racemised and recovered thus resulting in high yield and purity of the desired isomer.

The process of present invention allows obtaining the said intermediate (III) of tapentadol with tremendous yields, purities that too under environmentally acceptable conditions which is more convenient and more efficient than the previously known methods.

### SUMMARY OF THE INVENTION

The present invention relates to a novel stereospecific synthesis of (−)(2S,3S)-1-dimethylamino-3-(3-methoxyphenyl)-2-methyl pentan-3-ol an intermediate in the synthesis of tapentadol.

In one aspect, the present invention relates to a process for the preparation of (−)(2S,3S)-1-dimethylamino-3-(3-methoxyphenyl)-2-methyl pentan-3-ol of formula III

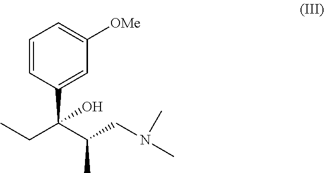

reacting the compound S(+)-1,1-dimethyl amino-2-methyl pentan-3-one of formula (V)

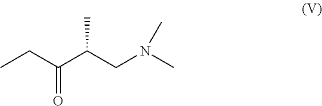

with the compound of formula (IV)

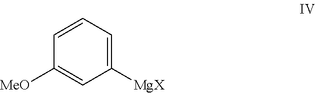

Where X is halogen (F, Cl, Br, I)
to afford the compound (−)(2S,3S)-1-dimethylamino-3-(3-methoxyphenyl)-2-methyl pentan-3-ol of formula (III).

In another aspect, the present invention relates to racemisation process for the conversion of (+) or (−)-1,1-dimethylamino-2-methyl pentan-3-one (V or Va) into the racemic compound comprising:
a) reacting (+) or (−)-1,1-dimethylamino-2-methyl pentan-3-one compound of formula (V or Va) with a base optionally in the presence of a solvent or a mixture of solvents; and,
b) recovering the racemic mixture compound (VI) in pure form.

### BRIEF DESCRIPTION OF THE DRAWING

FIG. 1: is a schematic representation of the process of the present invention.

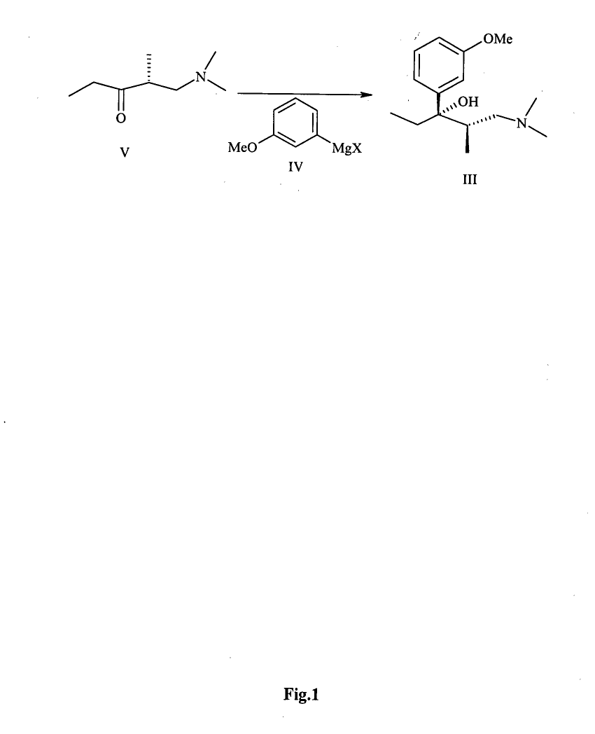

### DETAILED DESCRIPTION OF THE INVENTION

The present invention relates to a novel stereospecific synthesis of (−)(2S,3S)-1-dimethylamino-3-(3-methoxyphenyl)-2-methyl pentan-3-ol an intermediate in the synthesis of tapentadol.

In one embodiment, the present invention provides a process for the preparation of (−)(2S,3S)-1-dimethylamino-3-(3-methoxyphenyl)-2-methyl pentan-3-ol of formula III

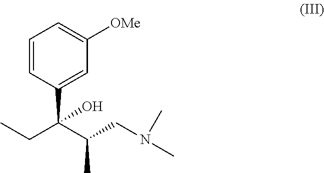

reacting the compound S(+)-1,1-dimethyl amino-2-methyl pentan-3-one of formula (V)

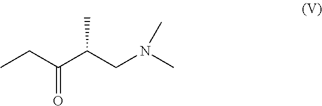

with the compound of formula (IV)

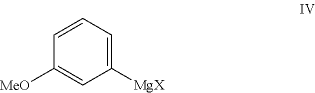

Where X is halogen (F, Cl, Br, I)
to afford the compound (−)(2S,3S)-1-dimethylamino-3-(3-methoxyphenyl)-2-methyl pentan-3-ol of formula (III).

The compound of formula (V) can also be prepared by the process described in the prior art for eg. Chirality 6: 389-399 (1994).

The reaction step of conversion of formula V to the intermediate compound of formula III comprises of subjecting the compound 3-bromo anisole to Grignard reaction consisting of magnesium, catalytic amount of iodine, and solvent ether to afford compound of formula IV followed by reaction with the compound of formula V to afford the compound of formula III.

The reaction step comprising Grignard reaction followed by condensation are carried out successively in a one pot.

The solvents employed is selected from the group consisting of hydrocarbons such as toluene, xylene, n-hexane, n-heptane, cyclohexane and the like; ethers such as tetrahydrofuran (THF), 1,4-dioxane, diethyl ether, diisopropyl ether and the like; halocarbonated solvents such as methylene chloride, ethylene chloride, chloroform, carbon tetrachloride, dichlorobenzene and the like; esters such as ethyl acetate, isopropyl acetate, n-butyl acetate, tert-butyl acetate and the like; or mixtures of thereof. Preferably tetrahydrofuran (THF) is being used.

The reaction temperatures and time in step a) should be suitable to bring the reaction to completion at a minimum time, without the production of unwanted side products or impurities. The reaction temperature can be from about 0° C. to about 100° C. or boiling point of the solvent(s) used. Preferably at about 25° C.

The reaction time can vary in dependence on various parameters, such as, for example, temperature, pressure, nature of the compound to be reacted or the nature of the reagent and solvent(s) employed, and can be determined for the process in question by the person skilled in the art using preliminary tests.

The time period can be from about 15 mins. to about 10 hours, preferably from about 30 mins. to about 2 hours.

The molar equivalent of compound of formula IV and reagent used can be from about 0.25 to about 7 molar equivalents on the weight of the compound of formula V taken. Preferably 3 moles.

This reaction step is highly stereoselective, because the optical purity of the intermediate compound of formula V is greater than about 99% by chiral HPLC.

The particular intermediate compounds of formula (V) and (III) can be purified by conventional methods known to the person skilled in the art.

The intermediate compound of formula (III) obtained by the above described process of present invention can be further converted into Tapentadol hydrochloride of formula I by processes described in the art. Illustratively, by the process described in U.S. Pat. No. USRE39593E which is incorporated herein by reference in its entirety.

The compound of formula (III) obtained by the process of present invention described above is useful as an intermediate in the synthesis of various active pharmaceutical ingredients. For ex. Tapendaol.

The process reported for the preparation of compound of formula (III) in the above mentioned reference U.S. Pat. No. USRE39593E is by chiral HPLC separation method which is tedious and not suitable on commercial scale, moreover results in the desired isomer with very low yield and purity.

In another embodiment, the present invention relates to racemisation process for the conversion of (+) or (−)-1,1-dimethylamino-2-methyl pentan-3-one (V or Va) into the racemic compound comprising:
a) reacting (+) or (−)-1,1-dimethylamino-2-methyl pentan-3-one compound of formula (V or Va) with a base optionally in the presence of a solvent or a mixture of solvents; and,
b) recovering the racemic mixture compound (VI) in pure form.

The base that can be used in step a) include organic base or inorganic base. Inorganic bases such as sodium hydroxide, potassium hydroxide, potassium tert-butoxide sodium carbonate, potassium carbonate, sodium bicarbonate, aqueous ammonia and the like; The organic bases that can be used include, but are not limited to triethylamine, tripropylamine, pyridine, diisopropylamine, diisopropylethylamine and the like or mixtures thereof. Preferably aqueous sodium hydroxide is being used

The solvent that can be used optionally include but are not limited to hydrocarbons such as toluene, xylene, n-hexane, n-heptane, cyclohexane and the like; halogenated solvents such as methylene chloride, ethylene chloride, trichloroethylene, chloroform, chlorobenzene, dichlorobenzene and the like; esters such as ethyl acetate, isopropyl acetate, n-butyl acetate, tert-butyl acetate and the like; ethers such as diethyl ether, diisopropyl ether, tetrahydrofuram, 1,4-dioxane, methyl tertiary butyl ether and the like; aprotic polar solvents such as dimethyl formamide (DMF), dimethyl sulfoxide (DMSO), acetonitrile or mixtures thereof. Preferably methylene chloride.

The reaction temperature range can be from about 30° C. to about 100° C., or boiling point of the solvents used. Preferably about 30° C.

The pH in the reaction step a) can be adjusted from about 7 to about 12, preferably about 12.

Advantageously, the process of present invention described herein has simple reaction steps, produces the intermediate surprisingly in high yields and purities than the processes reported in the literature and well amenable on commercial scale.

As used herein, the term “HPLC” refers to High-performance liquid chromatography.

As used herein, the term “% area by HPLC” refers to the area in an HPLC chromatogram of one or more peaks compared to the total area of all peaks in the HPLC chromatogram expressed in percent of the total area.

In this specification the term “racemic mixture” may include mixtures of enantiomers in ratios other than, as well as, a 50:50 mixture of R:S enantiomers (for example from 99:1 to 1:99). A particular process of the invention begins with a 50:50 mixture of enantiomers. The process may involve differing mixtures of enantiomers at various stages (including, but not limited to 50:50 mixtures). The term “racemisation” covers the conversion of an unresolved enantiomer into a mixture containing the enantiomer to be resolved.

The present invention provides a simple, ecofriendly, costeffective, reproducible, robust, commercially suitable process for preparation intermediate of tapentadol hydrochloride.

The following examples are provided to enable one skilled in the art to practice the invention and are merely illustrative of the invention. The examples should not be read as limiting the scope of the invention as defined in the claims, appended herewith.

### EXAMPLES

### Example-1

### Preparation of (−)(2S,3S)-1-dimethylamino-3-(3-methoxyphenyl)-2-methyl pentan-3-ol (III)

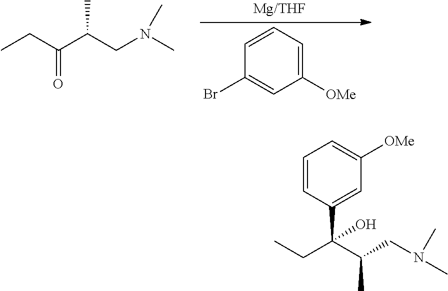

(S)-(+)-1,1-dimethylamino-2-methyl pentan-3-one (V) (42 gms) (SOR: +95°) was added to 3-Methoxyphenylmagnesium bromide (IV) (prepared from 166 gms of 3-Bromo anisole and 21.3 gms of magnesium in 126 ml of tetrahydrofuran) drop-wise for 90-120 min. at about 25° C. and maintained for 30 min. Then the resulted solution was cooled to 5° C. Then added water slowly at 5-10° C. Added 157 ml. of acetic acid to adjust the pH=4.0 at 5-10° C. The aqueous layer was basified (P^{H}=8.0) with 80 ml. of aqueous sodium hydroxide solution. The organic layer was separated. The aqueous layer was extracted with (3×200 ml.) of toluene and distilled under high vacuum to obtain a residue.

Wt: 92 gms. (% Yield: 99%); Purity by GC: 78.9%.

b) To 94 gms. of the residue obtained above, added 470 mL of water and the pH was adjusted to about 4 with 212 ml. of acetic acid. The layers were separated and the aqueous layer was washed with dichloromethane (4×50 ml.). Then, the aqueous layer P^{H} was adjusted to 12 with 180 ml. of aqueous sodium hydroxide solution at ambient temperature and the product was extracted from aqueous layer with ethyl acetate (4×100 ml.). Finally the ethyl acetate layer was distilled to obtain the title compound as residue.

Yield: 86 gms (% Yield: 94%); Purity by GC: 98%; SOR=(−) 20° (C=1% CH₃OH).

### Example-2

### Racemisation of S-(+)-1,1-dimethylamino-2-methyl pentan-3-one (V)

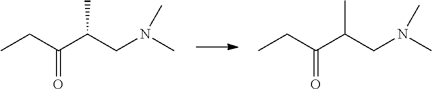

Taken 100 gms. of S-(+)-1,1-dimethylamino-2-methyl pentan-3-one (V) (SOR: +98°) (Purity by GC: 98%) and 930 ml. of water and added 80 ml. of aqueous sodium hydroxide solution (50%) and maintained for 30 min. followed by extracted with dichloromethane (4×100 ml). The organic layer was separated and distilled to provide the compound (VI).

Yield: 95 gms (% Yield: 95%); SOR: 0° (C=1% in CH₃OH).

### Example-3

### Racemisation of R-(−)-1,1-dimethylamino-2-methyl pentan-3-one (Va)

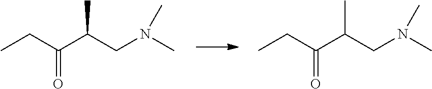

Taken 100 gms of R-(−)-1,1-dimethylamino-2-methyl pentan-3-one (Va) (SOR: −98°) (Purity by GC: 98%) and 930 ml. of water and added 80 ml. of aqueous sodium hydroxide solution (50%) and maintained for 30 min. followed by extracted with dichloromethane (4×100 ml). The organic layer was separated and distilled to provide the compound (VI).

Yield: 92 gms (% Yield: 92%); SOR: 0° (C=1% in CH₃OH).

1-8. (canceled)

9. A process for preparing (−)(2S,3S)-1-dimethylamino-3-(3-methoxyphenyl)-2-methyl pentan-3-ol compound of formula III 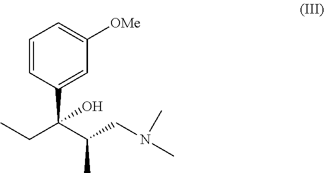
by reacting S(+)-1,1-dimethyl amino-2-methyl pentan-3-one of formula (V) 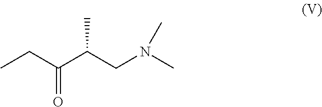
with a compound of formula (IV) 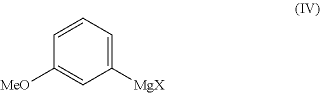
wherein X is a halogen (F, Cl, Br, I);
to produce a compound of formula (III).

10. The process of claim 9 wherein the solvent employed is selected from the group consisting of ethers, halo carbonated solvents, and esters.

11. The process of claim 10 wherein the ether is selected from the group consisting of tetrahydrofuran (THF), 1,4-dioxane, diethyl ether or mixtures thereof.

12. The process of claim 10 wherein the halo carbonated solvent is selected from the group consisting of methylene chloride, ethylene dichloride, chloroform, chlorobenzene, dichlorobenzene and mixtures thereof.

13. The process of claim 10 wherein the ester is selected from the group consisting of ethyl acetate, isopropyl acetate, n-butyl acetate, tert-butyl acetate and mixtures thereof.

14. The process of claim 9 wherein the solvent is tetrahydrofuran.

15. The process of claim 9 wherein the reaction is performed at a temperature from about 0° C. to about 100° C.

16. The process of claim 9 wherein the reaction is performed at the boiling point of the solvent(s) used.

17. The process of claim 9 wherein the reaction is performed at about 25° C.

18. The process of claim 9 wherein the reaction is carried out at a time period from about 15 minutes to about 10 hours.

19. The process of claim 9 wherein the reaction is carried out at a time period from about 30 minutes to about 2 hours.

20. The process of claim 9 wherein the molar equivalent of the compound of formula IV and reagent used is from about 0.25 to about 7 molar equivalents on the weight of the compound of formula V.

21. The process of claim 9 wherein the molar equivalent of the compound of formula IV and reagent used is about 1 molar equivalent on the weight of the compound of formula V.

22. The process of claim 9 wherein the compound of the formula III obtained has a purity greater than about 99% by chiral HPLC.

23. Use of an enantiomerically pure compound of the formula III to synthesize enantiomerically pure Tapentadol or a pharmaceutically acceptable acid addition salt thereof.

24. Use of the enantiomerically pure compound of formula V in the synthesis of enantiomerically pure Tapentadol or a pharmaceutically acceptable acid addition salt thereof.

25. A process for racemisation of (+) or (−)-1,1-dimethylamino-2-methyl pentan-3-one compound (V) (or) (Va) 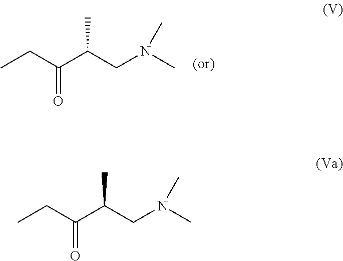
into a racemic compound of the formula (VI) 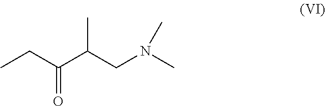
comprising:
a) reacting (+) or (−)-1,1-dimethylamino-2-methyl pentan-3-one compound of formula (V or Va) with a base, optionally in the presence of a solvent or a mixture of solvents; and,
b) recovering a racemic mixture compound (VI) in pure form.

26. The process of claim 25 wherein the base is selected from the group consisting of inorganic and organic bases and mixtures thereof.

27. The process of claim 25 wherein the base is selected from the group consisting of inorganic and organic bases and aqueous mixtures thereof.

28. The process of claim 26 wherein the base is selected from the group consisting of sodium hydroxide, potassium hydroxide, potassium tert-butoxide, sodium carbonate, potassium carbonate, sodium bicarbonate, aqueous ammonia and mixtures thereof.

29. The process of claim 26 wherein the organic base is selected from the group consisting of triethylamine, tripropylamine, pyridine, diisopropylamine, diisopropylethylamine and mixtures thereof.

30. The process of claim 25 wherein the base is sodium hydroxide.

31. The process of claim 25 wherein the solvent is selected from the group consisting of halogenated solvents, esters and mixtures thereof.

32. The process of claim 31 wherein the halogenated solvent is selected from the group consisting of methylene chloride, ethylene dichloride, chloroform, chlorobenzene, dichlorobenzene, and mixtures thereof.

33. The process of claim 31 wherein the ester is selected from the group consisting of ethyl acetate, isopropyl acetate, n-butyl acetate, tert-butyl acetate, and mixtures thereof.

34. The process of claim 25 wherein the solvent is methylene chloride.

35. The process of claim 25, wherein reaction step (a) is carried out at a temperature from about 30° C. to about 100° C.

36. The process of claim 25 wherein reaction step (a) is carried out at a temperature of the boiling point of the solvent(s) used.

37. The process of claim 25 wherein reaction step (a) is carried out at about 30° C.

38. The process of claim 25 wherein recovery of racemic compound of the formula VI is by extraction followed by distillation.
